SEQUENCE LISTING (2 sequences)

SEQ ID NO: 1 - protein, 18 aa, Lycopersicon Esculentum
  misc_feature: Xaa=hydroxyproline
RGANLPXXSXASSXXSKE

SEQ ID NO: 2 - protein, 18 aa, Lycopersicon Esculentum
  misc_feature: Xaa=hydroxyproline
NRKPLSXXSXKPADGQRP

(19) United States
(12) Patent Application Publication
(10) Pub. No.: US 2003/0211941 A1
(43) Pub. Date: Nov. 13, 2003

(54) Methods for identifying signalling molecules

(76) Inventors: Clarence A. Ryan, Pullman, WA (US); Gregory L. Pearce, Palouse, WA (US)

Correspondence Address: CHRISTENSEN, O'CONNOR, JOHNSON, KINDNESS, PLLC, 1420 FIFTH AVENUE, SUITE 2800, SEATTLE, WA, 98101-2347, US

(21) Appl. No.: 10/204,341

(22) PCT Filed: Feb. 13, 2001
(86) PCT No.: PCT/US01/05191

(51) Int. Cl. A01N 63/00; C07K 14/415

(52) U.S. Cl. 504/117; 530/370

(57) ABSTRACT

The present invention provides methods for identifying and isolating signalling molecules, such as signalling molecules that interact with a plant cell membrane receptor molecule. In the practice of the present invention, the ability of a sample of biological material to induce a pH change in a liquid plant cell culture is used as an assay for the presence of one or more signalling molecules in the biological material.

### FIELD OF THE INVENTION

The present invention relates to signalling molecules that mediate biological responses, and to biological receptors.

### BACKGROUND OF THE INVENTION

Many biological processes in plants, such as responses to pest and pathogen attack, are mediated by signalling molecules. Examples of signalling molecules include peptides, oligosaccharides, and fatty acid derivatives, such as jasmonic acid. Isolation of a signalling molecule that mediates a biological response provides the opportunity, for example, to utilize genetic engineering techniques to manipulate the biological response to produce plants having desirable properties, such as an enhanced resistance to pest or pathogen attack.

Signalling molecules are typically present at very low concentrations within plants, or within organisms (such as pathogenic fungi) that induce a biological response in plants. Consequently, the purification of a sufficiently large amount of a signalling molecule for chemical analysis is a daunting technical problem. Thus there is a need for methods that facilitate the identification and isolation of signalling molecules that elicit a biological response in plants.

### SUMMARY OF THE INVENTION

The present invention provides methods for identifying and isolating signalling molecules, such as signalling molecules that interact with a plant cell membrane receptor molecule. In the practice of the present invention, the ability of a sample of biological material to induce a pH change in a liquid plant cell culture is used as an assay for the presence of one or more signalling molecules in the biological material. A liquid plant cell culture is a culture in which plant cells are grown in a liquid medium.

In one embodiment, the present invention provides methods for isolating signalling molecules including the steps of incubating a liquid plant cell culture, including plant cells and supernatant, in the presence of an aliquot of a biological material; measuring a change of pH in the plant cell culture, the pH change being induced by the biological material; and at least partially purifying a signalling molecule (that is capable of inducing a pH change in a liquid plant cell culture) from the biological material. Typically, the pH change is measured in the supernatant of the liquid plant cell culture. The term “at least partially purifying”, and grammatical equivalents thereof, as used herein, means that the proportion of the signalling molecule(s) in the biological material is higher after purification than before purification. Preferably the signalling molecule(s) will be purified to at least 90% purity, more preferably to at least 95% purity, most preferably to at least 99% purity.

In another embodiment, the present invention provides methods for isolating signalling molecules, the methods including a plurality of purification steps, each of the plurality of purification steps including the steps of incubating a liquid plant cell culture, including plant cells and supernatant, in the presence of an aliquot of a biological material; measuring a change of pH in the incubated cell culture, the pH change being induced by the biological material; and at least partially purifying a signalling molecule (that is capable of inducing a pH change in a liquid plant cell culture) from the biological material. Typically, the pH change is measured in the supernatant of the liquid plant cell culture.

In another embodiment, the present invention provides methods for isolating signalling molecules, the methods including the steps of separating a biological material into at least two fractions having different chemical compositions; contacting a liquid plant cell culture, including plant cells and supernatant, with a portion of at least one of the fractions; measuring a change of pH in the contacted plant cell culture, the pH change being induced by the biological material, and at least partially purifying a chemical signalling molecule (that is capable of inducing a pH change in a liquid plant cell culture) from the biological material. Typically, the pH change is measured in the supernatant of the liquid plant cell culture.

In another embodiment, the present invention provides methods for isolating signalling molecules including the steps of contacting a plurality of liquid plant cell cultures, each culture including plant cells and supernatant, with a plurality of biological materials; measuring a change of pH in the contacted plant cell culture, the pH change being induced by the biological material, and at least partially purifying a signalling molecule (that is capable of inducing a pH change in a liquid plant cell culture) from the biological material. Typically, the pH change is measured in the supernatant of the liquid plant cell culture.

In another embodiment, the present invention provides signalling molecules prepared in accordance with the methods of the present invention, such as the polypeptide signalling molecules isolated as described in Example 1 and Example 2 herein. Thus, in one aspect, the present invention provides isolated polypeptides consisting of the amino acid sequence set forth in SEQ ID NO: 1 or SEQ ID NO: 2.

### BRIEF DESCRIPTION OF THE DRAWINGS

The foregoing aspects and many of the attendant advantages of this invention will become more readily appreciated as the same become better understood by reference to the following detailed description, when taken in conjunction with the accompanying drawings, wherein:

FIG. 1 is a schematic representation of one embodiment of the methods of the present invention.

FIG. 2A shows the elution profile of fractionated tobacco plant material eluted, as described in Example 1, from a semi-preparative, reversed-phase, C18 column and assayed for ability to induce alkalinization of tobacco liquid cell culture.

FIG. 2B shows the alkalinization profile of fractionated tobacco plant material eluted, as described in Example 1, from a semi-preparative, reversed-phase, C18 column and assayed for ability to induce alkalinization of tobacco liquid cell culture. Peaks I and II contained tobacco systemins I (SEQ ID NO: 1) and II, (SEQ ID NO: 2), respectively.

FIG. 3A shows the elution profile of tobacco systemin I (SEQ ID NO: 1) from a narrow bore reversed-phase C18 column, as described in Example 1 herein. The peak identified as Peak 1b is tobacco systemin I (SEQ ID NO: 1), and peak 1a is an analogue of tobacco systemin I in which Ala at position 3 is substituted with threonine at position 3.

FIG. 3B shows the alkalinization profile (assayed in tobacco cell culture) of fractions containing tobacco systemin I (SEQ ID NO: 1) eluted from the narrow bore reversed-phase C18 column, as described in Example 1.

FIG. 4A shows the elution profile of tobacco systemin in II (SEQ ID NO: 2) from a narrow bore reversed-phase C18 column, as described in Example 1 herein. The peak identified as Peak 1 contained most tobacco systemin II (SEQ ID NO: 2).

FIG. 4B shows the alkalinization profile (assayed in tobacco cell culture) of fractions containing tobacco systemin II (SEQ ID NO: 2) eluted from the narrow bore reversed-phase C18 column, as described in Example 1.

FIG. 5A shows the elution profile from a semi-preparative, reversed phase, C18 HPLC column of fractionated tobacco plant material containing the 5 kDa polypeptide disclosed in Example 2 herein. The arrows indicate the eluted fractions that contained most of the 5 kDa polypeptide.

FIG. 5B shows the alkalinization profile of fractionated tobacco plant material containing the 5 kDa polypeptide disclosed in Example 2 herein. The arrow indicates the eluted fraction that contained most of the 5 kDa polypeptide.

FIG. 6 shows a comparison of the ability of tobacco systemin I (TOB-I) (SEQ ID NO: 1), tobacco systemin II (TOB-II) (SEQ ID NO: 2), tomato systemin (TOM SYS) and the 5 kDa polypeptide (identified as AF in FIG. 6) disclosed in Example 2, to induce a pH increase in liquid plant cell cultures. Tobacco cell cultures were used to assay the alkalinization activity of tobacco systemin I (SEQ ID NO: 1), tobacco systemin II (SEQ ID NO: 2) and the 5 kDa polypeptide. Tomato systemin does not cause alkalinization of tobacco suspension cells.

FIG. 7A shows the alkalinization profile (using N. tabacum suspension cultured cells) of extracts from flower buds of mature tobacco plants.

FIG. 7B shows the alkalinization profile (using N. tabacum suspension cultured cells) of extracts from leaves of young tobacco plants

### DETAILED DESCRIPTION OF THE PREFERRED EMBODIMENT

The present invention provides methods for isolating signalling molecules. Signalling molecules are molecules that elicit a response from a biological cell, such as a plant cell. Representative examples of biological responses elicited by signalling molecules are growth, development, response to cellular damage, response to environmental stimuli and response to pest or pathogen attack. Examples of plant responses to pest or pathogen attack include the production of proteinase inhibitor proteins and other defensive proteins such as phytoalexins. Additionally, plant defense responses are reviewed, for example, by E. Kombrink and I. E. Somssich, “Defense Responses of Plants to Pathogens” in Advances in Biochemical Research 21:1-34 (1995), Academic Press. Signalling molecules isolatable by the methods of the present invention can be of microbial origin, such as those that microorganisms release in their various interactions with plants, such as pathogens, nitrogen fixers, saprophytes and micorhizzae.

Signalling molecules can be a chemical compound or chemical element. By way of non-limiting example, signalling molecules can be proteins, peptides, such as systemin, carbohydrates, such as plant cell wall fragments, volatile compounds, such as ethylene or methyl jasmonate, cyclic organic compounds such as auxins, cytokinins, gibberellins, abscisic acid, or chemical elements, such as metal ions.

Some signalling molecules interact with a receptor molecule which may be located in, or associated with, a cellular membrane, such as the plasma membrane that encloses the cell. Other signalling molecules interact with soluble receptors within the cell, or interact directly with genomic DNA.

In the practice of the present invention, cellular material, such as plant material (for example; leaves, stems, roots, flowers, seeds and storage organs) or microorganisms (such as pathogenic bacteria and fungi that cause plant disease, or symbiotic bacteria such as Rhizobium) is treated so as to yield a biological material that is to be tested for the presence of one or more signalling molecules. The cellular material may be treated to disrupt cells, for example by homogenizing the cellular material in a blender, or by grinding (in the presence of acid-washed, siliconized, sand if desired) the cellular material with a mortar and pestle, or by subjecting the cellular material to osmotic stress that lyses the cells. Cell disruption may be carried out in the presence of a buffer that maintains the contents of the disrupted cells at a desired pH, such as the physiological pH of the cells. The buffer may optionally contain inhibitors of endogenous, degradative enzymes, such as proteases and amylases.

The cellular material may also be treated in a manner that does not disrupt a significant proportion of cells, but which removes chemicals from the surface of the cellular material, and/or from the interstices between cells (or from the interstices within plant cell walls). For example, the cellular material can be soaked in a liquid buffer, or, in the case of plant material, can be subjected to a vacuum, in order to remove chemicals located in the intercellular spaces and/or in the plant cell wall. If the cellular material is a microorganism, the supernatant of the microorganism culture can be tested for its ability to cause a change in the pH of a liquid plant cell culture.

If the cellular material is disrupted in the presence of a buffer (hereinafter termed a homogenization buffer), then the homogenization buffer may include reductant, polyphenol inactivators, and protease inhibitors. Prior to large-scale purification of one or more signalling molecules from the biological material, trials can be performed to determine which components are required in the homogenization buffer. Labile compounds are preferably added to the buffer immediately before cellular disruption. Covalent inhibitors need only be present during disruption of the cellular material, while competitive inhibitors should be present at all stages of the purification of the signalling molecule(s). Examples of reductants that can be included in the homogenization buffer are dithiothreitol (DTT), for example at a working concentration of 2 to 5 mM, or 2-mercaptoethanol, for example at a working concentration of 14 mM. Other reductants include ascorbate and reduced glutathione.

When attempting to identify and isolate protein and/or peptide signalling molecules, it may be desirable to include one or more protease inhibitors in the homogenization buffer. Representative examples of protease inhibitors include: serine protease inhibitors (such as phenylmethylsulfonyl fluoride (PMSF), benzamide, benzamidine HCl, ε-Amino-n-caproic acid and aprotinin (Trasylol)); cysteine protease inhibitors, such as sodium p-hydroxymercuribenzoate; competitive protease inhibitors, such as antipain and leupeptin; covalent protease inhibitors, such as iodoacetate and N-ethylmaleimide; aspartate (acidic) protease inhibitors, such as pepstatin and diazoacetylnorleucine methyl ester (DAN); metalloprotease inhibitors, such as EGTA [ethylene glycol bis(β-aminoethyl ether) N,N,N′,N′-tetraacetic acid], and the chelator 1, 10-phenanthroline. The biological material may be ground in the homogenization buffer at a temperature of about 4° C. or less.

One of ordinary skill in the art will recognize that the composition of homogenization buffer, and the homogenization conditions, can readily be modified, without undue experimentation, to identify an extraction regime that permits extraction of one or more signalling molecules in biologically active form. Representative homogenization buffers and extraction regimes are set forth in Example 1 herein.

The biological material can be tested, without further purification or treatment, for its ability to induce a pH change in a liquid plant cell culture, provided it is not buffered to prevent an induced pH change from occurring, or the biological material can be further treated to generate fractions having different chemical compositions. The biological material can be fractionated by any art-recognized means. For example, biological material in liquid form (such as plant parts ground up in a homogenization buffer) can be treated to selectively precipitate certain chemical components, such as by dissolving ammonium sulfate in the liquid material (or by adding trichloroacetic acid) in order to selectively precipitate certain proteins. The precipitated material can be separated from the unprecipitated material by centrifugation, or by filtration. The precipitated material (whether or not resolubilized) and the unprecipitated material can then be tested for its ability to induce a pH change in a plant cell culture, or can be further fractionated if so desired.

By way of example, a number of different neutral or slightly acidic salts have been used to solubilize, precipitate, or fractionate proteins in a differential manner. These include NaCl, Na₂SO₄, MgSO₄ and NH₄(SO₄)₂. Ammonium sulfate is the precipitant used most frequently in the salting out of proteins. The biological material to be treated with ammonium sulfate should first be clarified by centrifugation. The biological material should be in a buffer at neutral pH unless there is a reason to conduct the precipitation at another pH; in most cases the buffer will have ionic strength close to physiological. Precipitation is usually performed at 0-4° C. and all solutions should be precooled to that temperature range.

The weight of solid ammonium sulfate needed to bring the volume of starting material to 80-85% saturation should be determined and the required amount of ammonium sulfate weighed out. The vessel containing the biological material should be equipped with a thermometer and a glass electrode for monitoring pH, and a suitable magnetic or motor-driven stirrer. Ammonium sulfate is added in increments with constant stirring and with adjustment of pH by addition of 1 N NH₄OH, as required. Each addition of salt is made only after the previously added amount has completely dissolved. When all of the salt has been added, the mixture is stirred for another 15 to 30 min to allow equilibration of the solvent and protein. The mixture is then centrifuged at about 10,000 g for 10 min, or 3000 g for 30 min, in a precooled centrifuge at 0-4° C. The supernatant fluid can be decanted or drawn off by suction.

Representative examples of other art-recognized techniques for purifying, or partially purifying, signalling molecules (including peptides and/or proteins) from biological material are exclusion chromatography, ion-exchange chromatography, hydrophobic interaction chromatography, reversed-phase chromatography and immobilized metal affinity chromatography.

Hydrophobic interaction chromatography and reversed-phase chromatography are two separation methods based on the interactions between the hydrophobic moieties of a sample and an insoluble, immobilized hydrophobic group present on the chromatography matrix. In hydrophobic interaction chromatography the matrix is hydrophilic and is substituted with short-chain phenyl or octyl nonpolar groups. The mobile phase is usually an aqueous salt solution. In reversed phase chromatography the matrix is silica that has been substituted with longer n-alkyl chains, usually C₈ (octylsilyl) or C₁₈ (octadecylsilyl). The matrix is less polar than the mobile phase. The mobile phase is usually a mixture of water and a less polar organic modifier.

Separations on hydrophobic interaction chromatography matrices are usually done in aqueous salt solutions, which generally are nondenaturing conditions. Samples are loaded onto the matrix in a high-salt buffer and elution is by a descending salt gradient. Separations on reversed-phase media are usually done in mixtures of aqueous and organic solvents, which are often denaturing conditions. In the case of protein and/or peptide purification, hydrophobic interaction chromatography depends on surface hydrophobic groups and is carried out under conditions which maintain the integrity of the protein molecule. Reversed-phase chromatography depends on the native hydrophobicity of the protein and is carried out under conditions which expose nearly all hydrophobic groups to the matrix, i.e., denaturing conditions.

Ion-exchange chromatography is designed specifically for the separation of ionic or ionizable compounds. The stationary phase (column matrix material) carries ionizable functional groups, fixed by chemical bonding to the stationary phase. These fixed charges carry a counterion of opposite sign. This counterion is not fixed and can be displaced. Ion-exchange chromatography is named on the basis of the sign of the displaceable charges. Thus, in anion ion-exchange chromatography the fixed charges are positive and in cation ion-exchange chromatography the fixed charges are negative.

Retention of a molecule on an ion-exchange chromatography column involves an electrostatic interaction between the fixed charges and those of the molecule, binding involves replacement of the nonfixed ions by the molecule. Elution, in turn, involves displacement of the molecule from the fixed charges by a new counterion with a greater affinity for the fixed charges than the molecule, and which then becomes the new, nonfixed ion.

The ability of counterions (salts) to displace molecules bound to fixed charges is a function of the difference in affinities between the fixed charges and the nonfixed charges of both the molecule and the salt. Affinities in turn are affected by several variables, including the magnitude of the net charge of the molecule and the concentration and type of salt used for displacement.

Solid-phase packings used in ion-exchange chromatography include cellulose, dextrans, agarose, and polystyrene. The exchange groups used include DEAE (diethylaminoethyl), a weak base, that will have a net positive charge when ionized and will therefore bind and exchange anions; and CM (carboxymethyl), a weak acid, with a negative charge when ionized that will bind and exchange cations. Another form of weak anion exchanger contains the PEI (polyethyleneimine) functional group. This material, most usually found on thin layer sheets, is useful for binding proteins at pH values above their pI. The polystyrene matrix can be obtained with quaternary ammonium functional groups for strong base anion exchange or with sulfonic acid functional groups for strong acid cation exchange. Intermediate and weak ion-exchange materials are also available. Ion-exchange chromatography need not be performed using a column, and can be performed as batch ion-exchange chromatography with the slurry of the stationary phase in a vessel such as a beaker. By way of example, in the practice of the present invention, polysulfoethyl aspartamide has been used as a cation exchanger (at pH 3) in HPLC as part of a purification protocol for plant polypeptide signalling molecules.

Gel filtration is performed using porous beads as the chromatographic support. A column constructed from such beads will have two measurable liquid volumes, the external volume, consisting of the liquid between the beads, and the internal volume, consisting of the liquid within the pores of the beads. Large molecules will equilibrate only with the external volume while small molecules will equilibrate with both the external and internal volumes. A mixture of molecules (such as peptides) is applied in a discrete volume or zone at the top of a gel filtration column and allowed to percolate through the column. The large molecules are excluded from the internal volume and therefore emerge first from the column while the smaller molecules, which can access the internal volume, emerge later. The volume of a conventional matrix used for protein purification is typically 30 to 100 times the volume of the sample to be fractionated. The absorbance of the column effluent can be continuously monitored at a desired wavelength using a flow monitor.

A technique that can be applied to the purification of signalling molecules is High Performance Liquid Chromatography (HPLC). HPLC is an advancement in both the operational theory and fabrication of traditional chromatographic systems. HPLC systems for the separation of biological macromolecules vary from the traditional column chromatographic systems in three ways; (1) the column packing materials are of much greater mechanical strength, (2) the particle size of the column packing materials has been decreased 5- to 10-fold to enhance adsorption-desorption kinetics and diminish bandspreading, and (3) the columns are operated at 10-60 times higher mobile-phase velocity. Thus, by way of non-limiting example, HPLC can utilize exclusion chromatography, ion-exchange chromatography, hydrophobic interaction chromatography, reversed-phase chromatography and immobilized metal affinity chromatography.

An exemplary purification strategy that has been found useful in the practice of the present invention for purifying plant signalling molecules is reverse phase, low pressure batch C₁₈ chromatography, followed by G-25 sephadex gel filtration, then further purification using HPLC. The pH is preferably kept low by the addition of 0.1% trifluoroacetic acid which can be removed by lyophilization. This approach was used to purify the plant signalling molecules described in Examples 1 and 2 herein.

Once a biological material has been prepared, a liquid plant cell culture is contacted with an aliquot of the biological material in order to test the ability of the biological material to induce a pH change in the plant cell culture. The cell culture can be derived from any species of plant, including gymnosperm or angiosperm plant species. Representative plant species include: tomato plants (including Lycopersicon esculentum and Lycopersicon peruvianum), potato plants, tobacco plants, alfalfa plants, maize plants and plants of the genus Arabidopsis. Art-recognized techniques for preparing and maintaining plant cell cultures are set forth in “Plant Cell Culture, A Practical Approach” (R. A. Dixon, ed.), Oxford University Press, 2^{nd} edition (1995), which publication is incorporated herein by reference. Representative plant cell culture techniques useful in the practice of the present invention are set forth in Example 4 herein.

A liquid plant cell culture is incubated in the presence of an aliquot of biological material for a period of time that is sufficient for a signalling molecule-induced change in the pH of the cell culture to occur. The required period of time can be readily determined by one of ordinary skill in the art, without undue experimentation, for example by conducting a time course experiment by adding equal aliquots of biological material to equal aliquots of a liquid plant cell culture, and measuring the pH of a cell culture aliquot at defined time intervals after addition of the biological material. In one embodiment of the invention, a liquid plant cell culture is incubated in the presence of an aliquot of biological material for from about one minute to about thirty minutes, more preferably from about five minutes to about ten minutes.

Some signalling molecules only induce a pH change in a liquid plant cell culture prepared from the same plant species from which the signalling molecule was isolated. Other signalling molecules induce a pH change in liquid plant cell cultures prepared from one or more plant species in addition to the plant species from which the signalling molecule was isolated. Typically, but not necessarily, when using the methods of the invention to isolate a novel signalling molecule, a liquid plant cell culture is utilized that is prepared from the same plant species from which the signalling molecule is isolated.

The pH of the plant cell culture can increase or decrease in response to the presence of a signalling molecule in the biological material. In some cases, the pH of the cell culture may transiently decrease (or increase) before increasing (or decreasing). The change can be measured by any art-recognized means for measuring pH, such as a pH meter that measures the conductivity of a solution as an indicator of pH, or a calorimetric assay, such as chemically-treated substrate (such as litmus paper), in which the pH of the material being assayed is indicated by the color of the substrate. Although any change in pH may be indicative of the presence of a signalling molecule in the biological material being tested, typically a change of at least 0.3 pH units, more preferably at least 0.8 pH units, most preferably at least 1.0 pH units, is considered indicative of the presence of a signalling molecule in the biological material.

While not wishing to be bound by theory, the change in pH of a liquid plant cell culture incubated in the presence of a biological material including a signalling molecule may be caused by interaction of the signalling molecule with a plasma membrane-bound receptor molecule that causes ion transport across the plasma membrane of the plant cell. Thus, for example, the import of hydrogen ions (H⁺ions) into the plant cells, and the simultaneous export of potassium ions (K⁺ions) will cause a decrease in the pH of the cell culture supernatant.

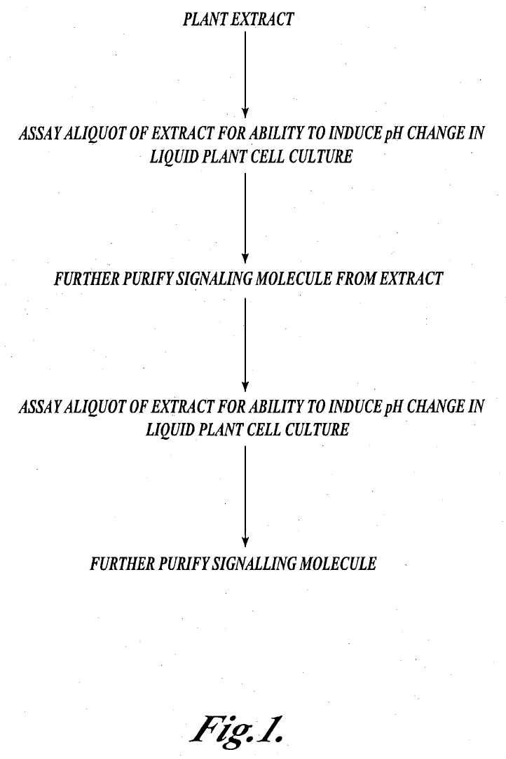

It will be understood that several rounds of assay (for the ability to cause a change in plant cell culture pH) and purification may be required in order to sufficiently purify a chemical signalling molecule to permit chemical characterization thereof, e.g., to permit determination of the amino acid sequence of a peptide signalling molecule. FIG. 1 shows a schematic representation of one embodiment of the present invention that utilizes several rounds of assay and purification of a plant extract to purify a signalling molecule from the extract. Any art-recognized technique (or combination thereof) can be used to further purify one or more signalling molecules from biological material. Examples of art-recognized protein purification techniques are set forth supra. Additionally, art-recognized techniques for the purification of proteins and peptides are set forth in Methods in Enzymology, Vol. 182, Guide to Protein Purification, Murray P. Deutscher, ed. (1990), which publication is incorporated herein by reference. Art-recognized techniques for the purification of carbohydrates are set forth in Methods in Enzymology 179(Part F): 3-422 (1989), which publication is incorporated herein by reference.

It is understood that in some cases there will be enough biological material remaining, after an aliquot has been removed to test its ability to induce a pH change in a liquid plant cell culture, to permit additional rounds of assay and purification to yield a completely pure, or substantially pure, signalling molecule capable of inducing a pH change in a liquid plant cell culture. In other cases, there will be insufficient biological material remaining, after an aliquot has been removed to test its ability to induce a pH change in a liquid plant cell culture, to permit further purification of a signalling molecule. In the latter situation, additional biological material can be prepared in the same manner as the previous batch of biological material that was capable of inducing a pH change in the plant cell culture. The new batch of biological material can then be subjected to further purification and the resulting material again tested for its ability to induce a pH change in the plant cell culture.

In another embodiment, the present invention provides methods for isolating signalling molecules including the steps of contacting a plurality of liquid plant cell cultures, each culture including plant cells and supernatant, with a plurality of biological materials; measuring a change of pH in the contacted plant cell culture, and at least partially purifying a chemical signalling molecule (that is capable of inducing a pH change in a liquid plant cell culture) from the biological material. By way of non-limiting example, the plurality of cell cultures can be contained within a plurality of separate containers, such as glass laboratory flasks or beakers, or within a container (or plurality of such containers) that defines a plurality of separate reservoirs, such as the wells of a microtitre plate. Thus, in one embodiment of the present invention, 1 ml of a liquid plant cell culture is placed into each well of a 24 well tissue culture plate, and from about 1 μl to about 20 μl of biological material to be assayed is added to each aliquot of liquid plant cell culture.

The methods of the present invention may be automated, for example by providing an automated dispensing means, which dispenses measured amounts of a biological material into a plurality of aliquots of a liquid plant cell culture, an automated incubation means which incubates the plant cell culture aliquots in the presence of the biological material under defined incubation conditions for a desired period of time, and an automated measuring means which measures the pH of the plant cell culture aliquots before and after (and optionally during) incubation of the cell culture aliquots in the presence of the biological material.

The following examples merely illustrate the best mode now contemplated for practicing the invention, but should not be construed to limit the invention.

### EXAMPLE 1

### Identification and Isolation of Tobacco Systemin Signalling Polypeptides Using the Methods of the Present Invention

Nine hundred 4-week-old tobacco plants were sprayed with methyl jasmonate and harvested 15 hours later in liquid nitrogen and stored at −20° C. until used (about 2 kg tissue). The frozen plants were blended with 2.8 L of 1% trifluoroacetic acid (TFA), filtered through 8 layers of cheesecloth and one layer of miracloth and centrifuged at 10,000× g for 20 min. The supernatant was poured through a column containing 100 g of Bakerbond™ Octadecyl (C18) 40 mm Prep LC packing equilibrated with 0.1% TFA/H₂O. The column was eluted under pressure of 8 psi using nitrogen gas. The 10-30% MEOH/0.1% TFA eluant was collected and lyophilized. The yield from each preparation averaged 0.91 g dry powder. The dry powder was dissolved in 5 ml 0.1% TFA and passed through a G-25 Sephadex column (4×35 cm) equilibrated with 0.1% TFA. The eluant was monitored at 280 nm, and a 10 μL aliquot from each tube was used for the alkalinization response assay using 2 ml cells for each assay.

Column fractions were assayed using suspension-cultured tobacco cells as follows. An aliquot of cells (usually 1 to 2 ml) were placed in the wells of culture plates, then aliquots from each column fraction (1 to 10 μL) were placed in each well of the culture plates. The cells in the plates were agitated by a circular motion of the rotary shaker on which they sat. The pH was then monitored over time and the change in pH at various times was plotted versus fraction number.

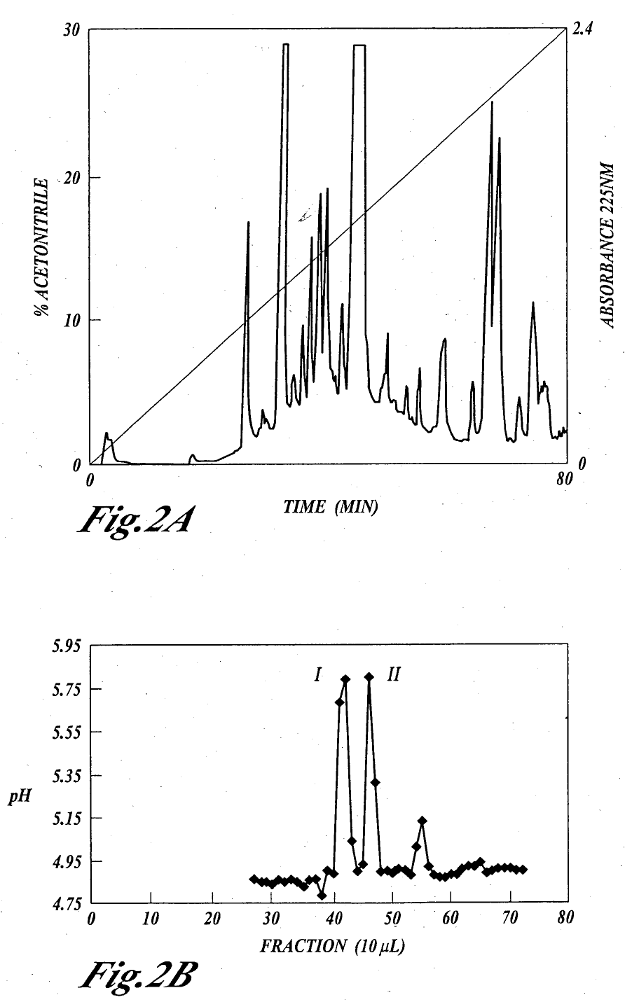

The ability to cause alkalinization of the cell culture was found in a broad peak eluting at one void volume, and the peak was pooled and lyophilized, yielding about 68 mg. Thirty mg of dry powder from the Sephadex G-25 column was dissolved in 0.1% TFA and injected into a semi-preparative reversed-phase C18 column (Vydac, Column 218 TP510, 10 by 250 mm, 5-μm beads, 300 angstrom pores). Solvent A consisted of 0.1% TFA in water. Solvent B was 0.1% TFA in acetonitrile. Samples were injected in solvent A and, after 2 min, a 90 min gradient to 30% solvent B was begun for elution. The flow rate was 2 ml/min and eluted peaks were monitored at 225 nm and assayed for alkalinization of L. peruvianum cell culture medium. The elution and alkalinization profiles are shown in FIG. 2A and FIG. 2B, respectively. Fractions containing peaks I and II were pooled and lyophilized.

SCX Chromatography was then performed on a poly Sulfo-Ethyl Asparamide column (The Nest Group, 4.6 by 200 mm, 5 μm beads) with the use of the following solvent systems: solvent A, 5 mM potassium phosphate, pH 3, in 25% acetonitrile; solvent B, 5-mM potassium phosphate, 500 mM potassium chloride in 25% acetonitrile, pH 3. Peak I was dissolved in 1 ml of solvent A, and applied to the column. After a 2 min wash with solvent A, a 90 min gradient to 30% B was applied. The flow rate was 1 mL/min, and the elution profile was monitored by absorbance at 214 nm. The fractions were assayed by alkalinization of tobacco cell culture medium. Fractions 61-62 were active and were pooled and lyophilized. Peak II was eluted with the same buffer system as peak I, but with a 90 min gradient to 100% B. The active fractions, 56 and 57, were pooled and lyophilized.

C18 chromatography of the two pooled peaks was performed at pH 6 on an analytical reversed-phase C18 column (Vydac, Column 219TP54, 4.6 by 250 mm, 5 μm pore size). Each peak was dissolved in 1 ml Solvent A (10 mM potassium phosphate, pH 6) and eluted with solvent B (10 mM potassium phosphate, pH 6, containing 50% acetonitrile). The same elution conditions as for the elution shown in FIG. 2A and FIG. 2B were employed. The flow rate was 1 ml/min and was monitored at 210 nm with the tobacco cell culture alkalinization assay. Active fractions (53-55 for the chromatography of peak I and 57-58 for the chromatography of peak II) were each pooled and lyophilized.

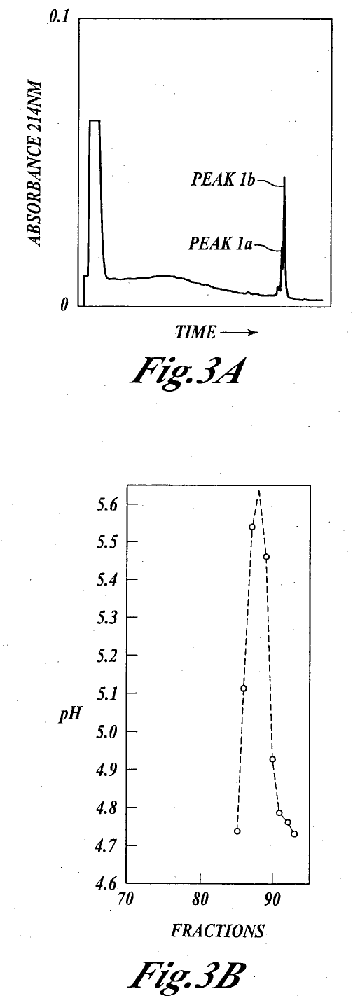

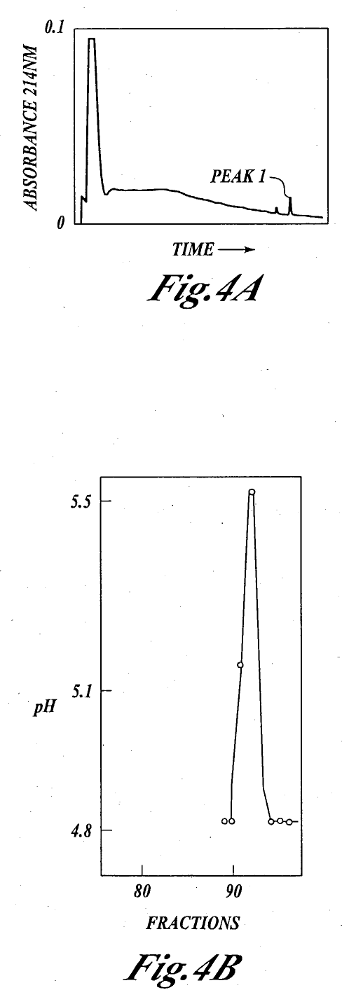

The two active peaks were solubilized in 1 ml 0.1% TFA each and injected into a narrow bore reversed-phase C18 column (Vydac, Column 218TP52, 2.1 by 250 mm, 5 μm beads, 300 angstrom pores). Solvent A consisted of 0.1% TFA in water, and solvent B was 0.05% TFA in methanol. Samples were injected in solvent A, and after 2 minutes, a 90 minute gradient from 0% solvent B to 30% solvent B was applied. The flow rate was 0.25 ml/min and the fractions (0.25 ml) were monitored at 214 nm and 2 μl were assayed for their alkalinization activity in tobacco cell cultures. The elution and alkalinization profiles for peak I activity are shown in FIG. 3A and FIG. 3B, respectively. The elution and alkalinization profile for peak II activity are shown in FIG. 4A and FIG. 4B, respectively.

The purified polypeptides were sequenced and named tobacco systemin I (peak I activity) (SEQ ID NO: 1) and tobacco systemin II (peak II activity) (SEQ ID NO: 2). Both are polypeptides of 18 amino acids and both are glycosylated. Using mild acid hydrolysis, the carbohydrates were removed, and the masses of both tobacco systemin I (SEQ ID NO: 1) and tobacco systemin II (SEQ ID NO: 2) were analyzed before and after acid hydrolysis using a MALDI-MS. The amino acid sequence of tobacco systemin I is NH₂-RGANLPXXSXASSXXSKE-COO. (SEQ ID NO:1). The amino acid sequence of tobacco systemin II is NH₂-NRKPLSXXSXKPADGQRP-COO (SEQ ID NO:2). The one letter abbreviation “X” represents hydroxyproline. The masses after acid hydrolysis exactly matched the masses obtained by sequence analysis. The loss of carbohydrate indicated that 9 pentose units were present in tobacco systemin I (SEQ ID NO: 1), while 6 units were in tobacco systemin II (SEQ ID NO: 2). Both peaks were active at low picomole levels in inducing tobacco proteinase inhibitor protein to accumulate when supplied to young tobacco plants through their cut stems, and in causing the alkalinization of tobacco suspension cell cultures (Table 1). The potency of the tobacco systemins was similar to that found for tomato systemin (disclosed in U.S. Pat. No. 5,378,819) in its alkalinization response in tomato cell suspension cultures and in inducing proteinase inhibitors in leaves of excised tomato plants (Table 1).

TABLE 1
CONCENTRATIONS FOR 1/2 MAXIMAL ACTIVITY IN

| Proteinase Inhibitor Inducing | | Alkalinization Assays |
|---|---|---|
| Assays (Tobacco Leaves) | | (Tobacco Cell Suspension Cultures) |
| fmol/plant | | fmol/2 ml |
| Tob Sys I (SEQ ID NO: 1) | ˜200 | ˜500 |
| Tob Sys II (SEQ ID NO: 2) | ˜200 | ˜500 |

Using prior art methods, the initial isolation of tomato systemin from tomato leaves took 4 years due to the tedious assays of young tomato plants for the induction of proteinase inhibitors in individual column fractions containing the partially purified tomato systemin. The present inventors worked on tobacco systemin for over 8 years without obtaining a pure tobacco systemin preparation. Utilizing the methods of the present invention, the inventors took approximately three months to purify the two tobacco systemins (SEQ ID NO: 1 and SEQ ID NO: 2) described herein.

### EXAMPLE 2

### Identification and Isolation of a 5 kDa Polypeptide From Tobacco that is Regulated by Cytokinins

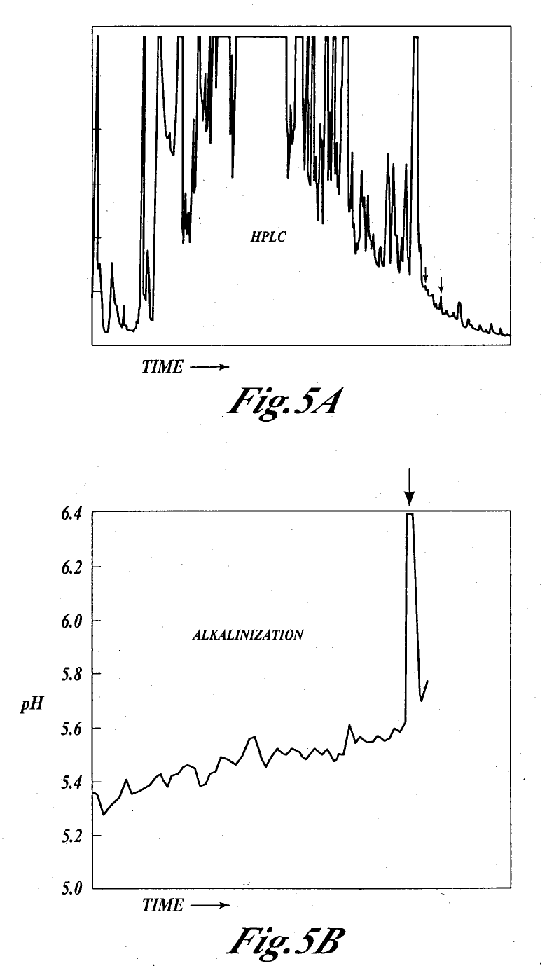

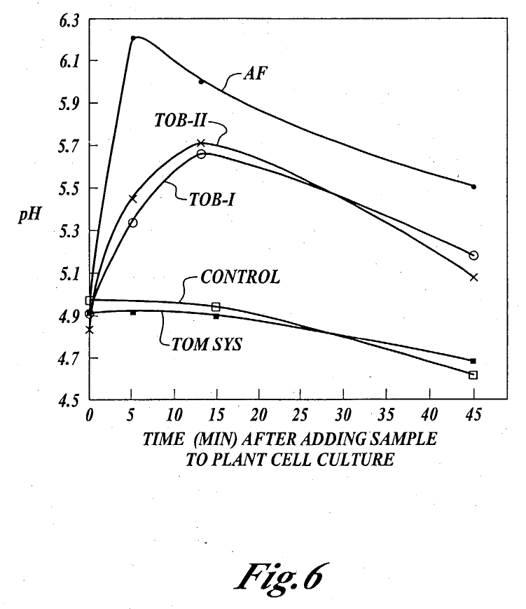

During the isolation of tobacco systemin I (SEQ ID NO: 1) and tobacco systemin II (SEQ ID NO: 2) described in Example 1 herein, fractions eluting much later than the two systemins were found to exhibit a strong alkalinization response (FIGS. 5 and 6). This peak was easily purified in a similar manner as described above for tobacco systemins I (SEQ ID NO: 1) and II (SEQ ID NO: 2). Sequence analysis and mass spectral analyses of the fraction revealed that it was a 5 kDa polypeptide. The pure polypeptide is more active (active at low pmole levels) than tobacco systemins (SEQ ID NO: 1 and SEQ ID NO: 2) in the liquid plant cell culture alkalinization assay, and it induces MAP kinase activities similar to systemins (see Example 5 below). However, it does not induce proteinase inhibitors in tobacco, indicating that it is not a systemin, but a new class of polypeptide signal in plants.

### EXAMPLE 3

### Identification of Tissue-Specific and/or Developmental Stage-Specific Polypeptide Signals

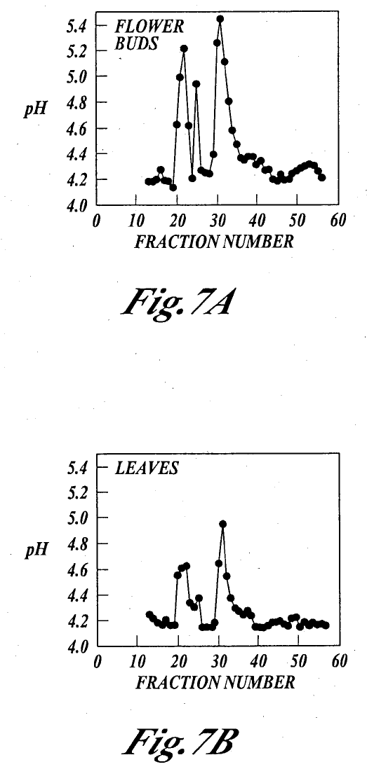

Another use of the methods of the present invention is to identify biologically active polypeptides in different tissues at different developmental stages. FIGS. 7A and 7B show the alkalinization profiles of extracts from flower buds and leaves, respectively, of young tobacco plants using N. tabacum suspension cultured cells. The extracts were taken through the same purification steps as the tobacco extracts shown in FIG. 2 that were used for tobacco systemin isolation. It can be seen that several peaks of activity are detected, some strong responses, others weak. These peaks can be readily isolated and characterized

### EXAMPLE 4

### Cell Suspension Culture

Cell suspension cultures useful in the methods of the invention can be prepared in the following manner. The initial cells are obtained from clean tissue, such as seeds (such as tomato, tobacco, potato, Arabidopsis and alfalfa seeds). The seeds are soaked in 50% bleach, 0.1% wetting agent (such as Tween 20), for 30 minutes (or for 45 minutes if the seeds appear to be dirty). The seeds are plated out on MSB medium for germination. When the seedlings have grown to the desired size, explants can be cut from a desired tissue type (e.g., cotyledons, hypocotyls or roots) or the seedlings can be cut up into 4-5 mm long pieces with a scalpel. The cut tissue is plated onto MST-12 medium. Good quality callus is transferred monthly to fresh MST-12 plates until friable (i.e., the callus is composed of a loose, fine association of cells) and uniform. To initiate a cell suspension culture, transfer as much as half the culture volume of callus to the liquid culture. The more friable calli are desirable for making a liquid culture.

The composition of MSB medium is: MS salts, Nitsch vitamins, 3% sucrose, 0.8% agar (pH 5.8). The composition of MST-12 medium is the same as MSB medium but with the addition of 0.1 mg/L benzyl adenine, 2.0 mg/L 2,4-D, 1 to 2 grams of casein hydrolysate (pH 5.8).

The cells can be maintained in 125 mL Ehrlenmeyer flasks on an orbital shaker (160 rpm) under constant light. Three milliliters of cells are subcultured every seven days into 45 mL of sterile media (unbuffered, pH 5.5 adjusted with 0.1 M KOH) containing 3% sucrose, 4.3 g/L Murashige and Skoog salt mixture, 5 mg/L 1-napthylacetic acid, 2 mg/L 6-benzylaminopurine, 110 mg/L Nitsch and Nitsch vitamin powder, 1 mg/L thiamine, 100 mg/L myo-inositol and 1 mM EDTA. Cells can be used for alkalinization assays from 4 to 8 days after subculturing.

A representative tomato cell line useful in the practice of the present invention is Lycopersicon peruvianum cell line Msk8.

### EXAMPLE 5

### MAP Kinase Activation of Tobacco Systemin I (SEO ID NO: 1), Tobacco Systemin II (SEQ ID NO: 2) and the 5 kDa Polypeptide

The ability of tobacco systemin I (SEQ ID NO: 1), tobacco systemin II (SEQ ID NO: 2) and the 5 kDa polypeptide, described in Example 2 herein, to stimulate the phosphorylation of a MAP kinase protein in tobacco leaf extracts was investigated. The MAP kinase assay utilized was essentially as described in Stratmann, J. W. and Ryan, C. A., Proc. Nat'l. Acad. Sci. U.S.A. 94:11085-11089 (1997), which publication is incorporated herein by reference. Tobacco systemin U (SEQ ID NO: 2) and the 5 kDa polypeptide each possess MAP kinase stimulating activity.

While the preferred embodiment of the invention has been illustrated and described, it will be appreciated that various changes can be made therein without departing from the spirit and scope of the invention.

The embodiments of the invention in which an exclusive property or privilege is claimed are defined as follows:

1. A method for isolating a signalling molecule comprising the steps of:
(a) incubating a liquid plant cell culture, including plant cells and supernatant, in the presence of an aliquot of a biological material;
(b) measuring a change of pH in the incubated cell culture; and
(c) at least partially purifying a chemical signalling molecule from the biological material, said chemical signalling molecule being capable of inducing a pH change in a liquid plant cell culture.

2. The method of claim 1 wherein said plant cells are selected from the group consisting of tomato plant cells, potato plant cells, tobacco plant cells and alfalfa plant cells.

3. The method of claim 1 wherein said plant cell culture is incubated in the presence of said aliquot of biological material for from about 1 minute to about 30 minutes.

4. The method of claim 3 wherein said plant cell culture is incubated in the presence of said aliquot of biological material for from about 5 minutes to about 10 minutes.

5. The method of claim 1 wherein said pH increases in response to the biological material.

6. The method of claim 5 wherein said pH increases by at least about 0.3 pH units in response to the biological material.

7. The method of claim 6 wherein said pH increases by at least about 0.8 pH units in response to the biological material.

8. A method for isolating a signalling molecule comprising:
a plurality of purification steps, each of said plurality of purification steps comprising the steps of:
(a) incubating a liquid plant cell culture, including plant cells and supernatant, in the presence of an aliquot of a biological material;
(b) measuring a change of pH in the contacted cell culture; and
(c) at least partially purifying a chemical signalling molecule from the biological material, said chemical signalling molecule being capable of inducing a pH change in a liquid plant cell culture.

9. A method for isolating a signalling molecule comprising the steps of:
(a) separating a biological material into at least two fractions having different chemical compositions;
(b) contacting a liquid plant cell culture, including plant cells and supernatant, with a portion of at least one of the fractions;
(c) measuring a change of pH in the contacted plant cell culture; and
(d) at least partially purifying a chemical signalling molecule from the biological material, said chemical signalling molecule being capable of inducing a pH change in a liquid plant cell culture.

10. A method for isolating a signalling molecule comprising the steps of:
(a) contacting a plurality of liquid plant cell cultures, each culture including plant cells and supernatant, with a plurality of biological materials;
(b) measuring a change of pH in the contacted plant cell culture; and
(c) at least partially purifying a chemical signalling molecule from the biological material, said chemical signalling molecule being capable of inducing a pH change in a liquid plant cell culture.
